(19) United States
(12) Patent Application Publication
(10) Pub. No.: US 2003/0176886 A1
(43) Pub. Date: Sep. 18, 2003

(54) Vascular catheter with expanded distal tip for receiving a thromboembolic protection device and method of use

(76) Inventors: Mark H. Wholey, Oakmont, PA (US); Petra Wholey, San Antonio, TX (US); Michael H. Wholey, San Antonio, TX (US)

Correspondence Address: Timothy D. Broms, Pietragallo, Bosick & Gordon, One Oxford Centre, 38th Floor, 301 Grant Street, Pittsburgh, PA, 15219, US

(21) Appl. No.: 10/348,137

(22) Filed: Jan. 21, 2003

Related U.S. Application Data
(60) Provisional application No. 60/363,310, filed on Mar. 12, 2002.

(51) Int. Cl. A61M 29/00

(52) U.S. Cl. 606/200

(57) ABSTRACT

A vascular catheter including a radially expandable segment, such as an inflatable balloon, and an expanded distal tip having an increased storage volume is disclosed. The inflatable balloon segment of the catheter may be used to provide traditional balloon angioplasty to a portion of a blood vessel narrowed by stenosis, and the expanded distal tip of the catheter apparatus may be used to safely capture, store, and remove a thromboembolic protection device such as an embolic filter used to catch pieces of plaque and other embolic material dislodged during the balloon angioplasty procedure. The catheter of the present invention provides an effective means for dilating a narrowed portion of a blood vessel, as well as preventing the need for deploying a second catheter system to capture and retrieve the embolic filter. The present invention also greatly reduces the chance for plaque and other thromboembolic material to escape from the embolic filter and enter the patient's bloodstream.

### CROSS-REFERENCE TO RELATED APPLICATION

This application claims the benefit of U.S. Provisional Patent Application Serial No. 60/363,310 filed Mar. 12, 2002.

### FIELD OF THE INVENTION

The present invention relates to transluminal angioplasty, and more particularly relates to a vascular catheter for providing balloon angioplasty while at the same time providing improved thromboembolic protection. Methods of utilizing the catheter apparatus to provide balloon angioplasty and thromboembolic protection are also provided.

### BACKGROUND INFORMATION

It is common practice today to open occluded (i.e. blocked) or stenotic (i.e. narrowed) blood vessels by inserting a guide wire and then a catheter carrying a balloon shaped segment and inflating the balloon, which exerts a radial force to press stenosis outward against the wall of the blood vessel. This procedure is called balloon angioplasty. Frequently, an implantable metallic stent will also be used to provide greater radial strength at the stenotic portion of the blood vessel, and to provide longer-term patency.

In order to help deliver balloon catheters and stent devices, special guiding catheters or sheaths are often used. These guiding catheters or sheaths are placed away (or upstream) from the targeted lesion or stenotic area. A guide wire may be advanced past the stenotic area, allowing the subsequent balloon catheters and stents to be advanced through the guiding catheter or sheath to the target area of the blood vessel.

During the balloon angioplasty procedure and stent placement at the stenotic lesion, there may exist the risk of dislodging fragments of plaque, thrombus (blood clots) and/or other material. These fragments may become dislodged from the stenotic lesion when the balloon segment is inflated. If the lesion involves arterial circulation, then the dislodged particles could flow into smaller vessels in the brain, other organs, or extremities, resulting in disastrous complications. Likewise, if the lesions involve the venous circulation, then the dislodged fragments could flow into the heart and lungs, possibly resulting in the demise of the patient.

Embolic protection devices are typically used to provide protection from such dislodged fragments of plaque and thrombus. These protection devices often consist of a small umbrella-like filter or lasso-shaped device attached to the end of a guide wire. The guide wire with the filter may be advanced across a stenotic lesion in an unexpanded state and then may be expanded in an area of the blood vessel past the stenotic lesion or downstream therefrom. When expanded, the filter can capture dislodged particles while still allowing blood to freely flow. The filter may stay expanded during all major parts of the procedure including pre-dilation of the stenotic lesion with a small balloon catheter, advancement and deployment of a stent, and post dilation with a large balloon catheter. When the procedure is completed, often a separate retrieval catheter will be advanced through the stented artery and be used to collapse and retrieve the embolic protection device.

There are many disadvantages to the retrieval catheters that are often used to collapse and remove embolic protection filters and other devices. If the targeted blood vessel is tortuous and the newly placed stent is at an angle, it is often difficult to pass a retrieval catheter into position to effectively and safely collapse the embolic filter. The distal tip of the retrieval catheter may often become snagged or caught on the edge of the stent as the retrieval catheter attempts to pass through the newly placed stent. Since retrieval catheters are usually straight, it is also often difficult to turn and advance off of obstructions, such as a newly placed stent.

Since a retrieval catheter usually requires a lumen that is larger than the dimensions of a filter wire, the retrieval catheter may cause scraping and/or focal dissection of the blood vessel wall as it passed through the diseased portion of the blood vessel.

Often the distal lumen of a retrieval catheter will be too small to safely collapse, store, and remove an embolic protection device. A partially collapsed filter or a filter not properly stored is at high risk for catching upon the edges of the newly placed stent as the retrieval catheter is removed, and/or for causing the embolic filter material to accidentally become removed from the support struts of the filter. As a result, the captured plaque and other thrombus may become free from the filter and enter into the blood stream. Moreover, the use of a retrieval catheter is an additional procedure that must be performed, requiring removal of the post-dilation balloon catheter and subsequent advancement of the retrieval catheter.

A need exists for a catheter that serves the dual purpose of providing balloon angioplasty to a stenotic lesion of a blood vessel, while at the same time providing an effective means for safely collapsing, storing, and removing an embolic protection filter or other device containing dislodged plaque and thromboembolic material.

The present invention has been developed in view of the foregoing, and to address other deficiencies of the prior art.

### SUMMARY OF THE INVENTION

The invention relates to an apparatus and method for providing balloon angioplasty while at the same time providing improved thromboembolic protection. The apparatus includes an inflatable balloon segment for providing balloon angioplasty and a distal tip with an increased volume which can safely and effectively store a thromboembolic protection device, such as an embolic filter, filled with embolic material, such as plaque or thrombus. The apparatus of the present invention can be advanced along a guide wire to provide balloon angioplasty to a stenotic portion of a blood vessel, and can then be further advanced along the guide wire to retrieve and store an embolic filter filled with embolic material in the expanded distal tip of the apparatus. The apparatus of the present invention may be advanced coaxially along a guide wire in a monorail system, or may be used in a standard over-the-wire system, both of which are well known in the art.

An aspect of the present invention is to provide a vascular catheter including a shaft having an expanded distal tip structured and arranged to receive at least a portion of a thromboembolic protection device, and a radially expandable segment disposed on the shaft.

Another aspect of the present invention is to provide a catheter assembly including a shaft having an expanded distal tip, a radially expandable segment disposed on the shaft, and a thromboembolic protection device at least partially receivable in the expanded distal tip.

A further aspect of the present invention is to provide a vascular catheter including a shaft having an expanded distal tip for storing at least a portion of a thromboembolic protection device, and a radially expandable segment disposed on the shaft.

Another aspect of the present invention is to provide a method of dilating blood vessels and protecting a patient from embolic material including the steps of inserting a guide wire including a thromboembolic protection device into a blood vessel and guiding the guide wire and the thromboembolic protection device past a stenotic portion of the blood vessel, expanding the thromboembolic protection device, guiding a catheter into the blood vessel along the guide wire, wherein the catheter includes a shaft having an expanded distal tip and a radially expandable segment, expanding the segment to dilate the stenotic portion of the blood vessel, guiding the catheter further along the guide wire to receive at least a portion of the thromboembolic protection device within the expanded distal tip, and removing the catheter and the thromboembolic protection device from the blood vessel.

A further aspect of the present invention is to provide a method of dilating blood vessels and protecting a patient from embolic material including the steps of inserting a guide wire including a thromboembolic protection device into a blood vessel and guiding the guide wire and the thromboembolic protection device past a stenotic portion of the blood vessel, expanding the thromboembolic protection device, guiding a catheter into the blood vessel along the guide wire, wherein the catheter includes a shaft having an expanded distal tip and a radially expandable segment, expanding the segment to dilate the stenotic portion of the blood vessel, retracting the guide wire until at least a portion of the thromboembolic protection device is received within the expanded distal tip, and removing the catheter and the thromboembolic protection device from the blood vessel.

These and other aspects of the present invention will be more apparent from the following description.

### BRIEF DESCRIPTION OF THE DRAWINGS

FIG. 1 is a partially schematic longitudinal sectional view of a catheter apparatus in accordance with an embodiment of the present invention.

FIG. 2 is a longitudinal side view of the apparatus of FIG. 1.

FIG. 3 is a sectional view taken along the line 3-3 of the apparatus of FIG. 2.

FIG. 4 is a sectional view taken along the line 4-4 of the apparatus of FIG. 2.

FIG. 5 is a longitudinal side view of the apparatus of FIG. 1, with the balloon segment in a deflated position.

FIG. 6 is a partially schematic longitudinal sectional view of a proximal end of a catheter apparatus in accordance with an embodiment of the present invention.

FIG. 7 is a longitudinal side view of the apparatus of FIG. 1 shown in conjunction with a guide wire and a thromboembolic protection device mounted on the guide wire.

FIG. 8 is a longitudinal side view of the apparatus of FIG. 1 shown in conjunction with a guide wire and a thromboembolic protection device mounted on the guide wire, with the thromboembolic protection device being retracted into the distal end of the catheter apparatus.

FIG. 9 is a longitudinal side view of the apparatus of FIG. 1 shown in conjunction with a guide wire and a thromboembolic protection device mounted on the guide wire, with the thromboembolic protection device retracted and partially stored within the distal end of the catheter apparatus.

FIG. 10 is a partially schematic longitudinal side view of a catheter apparatus in accordance with another embodiment of the present invention.

FIG. 11 is a sectional view taken along the line 11-11 of the apparatus of FIG. 10.

FIG. 12 is a sectional view taken along the line 12-12 of the apparatus of FIG. 10.

FIG. 13 is a partially schematic longitudinal side view of a catheter apparatus in accordance with another embodiment of the present invention.

FIG. 14 is a sectional view taken along the line 14-14 of the apparatus of FIG. 13.

FIG. 15 is a sectional view taken along the line 15-15 of the apparatus of FIG. 13.

FIG. 16 is a sectional view taken along the line 16-16 of the apparatus of FIG. 13.

FIG. 17 is a partially schematic longitudinal side view of a catheter apparatus in accordance with another embodiment of the present invention.

FIG. 18 is a sectional view taken along the line 18-18 of the apparatus of FIG. 17.

FIG. 19 is a sectional view taken along the line 19-19 of the apparatus of FIG. 17.

FIG. 20 is a longitudinal sectional view of the apparatus of FIG. 17.

FIG. 21 shows the apparatus of FIG. 1 being used to treat a stenosis of a blood vessel. FIG. 21 also shows the apparatus of FIG. 1 being used in conjunction with a guide wire and a thromboembolic protection device mounted on the guide wire, the thromboembolic protection device being in a substantially open position.

FIG. 22 shows the apparatus of FIG. 1 being used to treat a stenosis of a blood vessel in accordance with an embodiment of the present invention. FIG. 22 shows that a stent has been placed in the stenotic portion of the blood vessel, the catheter apparatus has been advanced further along the guide wire towards the thromboembolic protection device, and the thromboembolic protection device is beginning to collapse into the distal tip of the catheter apparatus.

FIG. 23 shows the apparatus of FIG. 1 being used to treat a stenosis of a blood vessel in accordance with an embodiment of the present invention. FIG. 23 shows that the thromboembolic protection device has been collapsed and partially stored within the distal tip of the apparatus of FIG. 1, and the apparatus is being removed from the blood vessel.

### DETAILED DESCRIPTION OF THE INVENTION

The apparatus of the present invention includes a catheter with a radially expandable segment, such as an inflatable balloon disposed on the shaft of the catheter, and an expanded distal tip that houses a thromboembolic protection device. As used herein, the term “expanded distal tip” means a distal portion of a shaft of a catheter that has a larger interior storage volume when compared a proximal portion of the shaft. This larger volume allows the expanded distal tip to effectively capture, store, and remove a thromboembolic protection device from a patient.

In the exemplary embodiments described herein, the catheter may be used in conjunction with a guide wire having the thromboembolic protection device attached near the end of the guide wire. The protection device may be extendable outward toward the interior wall of a blood vessel of a patient to trap embolic material typically broken lose by dilation or stenting of a stenotic portion of a blood vessel. As used herein, the term “thromboembolic protection device” includes filters, strainers, lassos, nets, traps, or any other assembly or device capable of capturing embolic material during an interventional procedure such as transluminal angioplasty or stenting. Embolic material includes plaque, thrombus, thromboembolic fragments, or any other material that may be dislodged from a blood vessel or released into the blood stream during an interventional procedure such as transluminal angioplasty.

In a preferred form of the invention, the guide wire and thromboembolic protection device combination may be inserted into a blood vessel to be treated, and the thromboembolic protection device may be extended outward to a substantially open position. The catheter of the present invention may then be advanced along the guide wire, and the radially expandable segment of the catheter may be used to dilate and provide stent placement to a stenotic portion of the blood vessel, as is commonly known in the art. When the procedure is completed and embolic material has collected in the thromboembolic protection device, the catheter of the present invention may be advanced further along the guide wire until the expanded thromboembolic protection device substantially meets the distal tip of the catheter. The thromboembolic protection device may then be collapsed and pulled into the expanded distal tip of the catheter via the guide wire, or the catheter may be advanced further along the guide wire until the collapsed protection device is sufficiently stored within the distal tip. The expanded distal tip of the catheter has a volume which is capable of safely and effectively storing the thromboembolic protection device filled with embolic material. The catheter and the collapsed thromboembolic protection device may then safely be removed from the blood vessel of the patient together, as a unit.

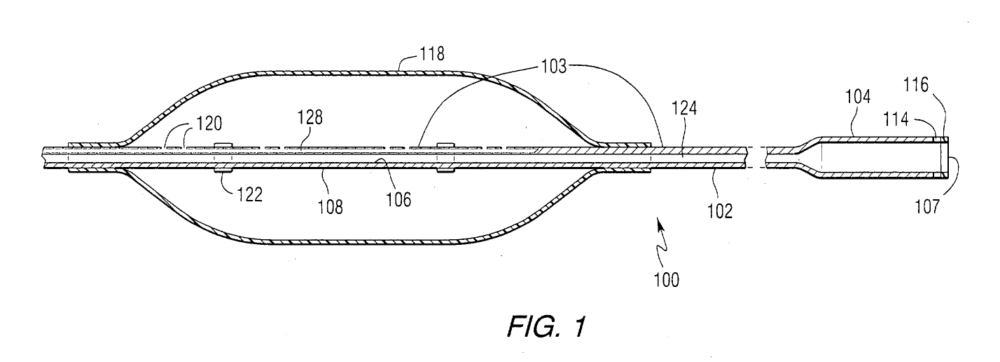

FIG. 1 is a partially schematic longitudinal sectional view of a vascular catheter 100 in accordance with an embodiment of the present invention. The catheter 100 may include a shaft 102, and the shaft includes an intermediate portion 103 and an expanded distal tip 104. The shaft of the catheter apparatus 100 may be made out of any suitable material, such as polyethylene, polyamide, polytetraflurethylene, or any other polyester compounds. In this embodiment, the expanded distal tip may be substantially cylindrical shaped, as shown in FIG. 1. FIG. 1 also illustrates that the transition from the intermediate portion of the shaft 103 to the expanded distal tip 104 may be a substantially gradual and substantially smooth transition. FIG. 1 shows that the shaft 102 includes inner wall 106 and outer wall 108. As shown in FIG. 1, the expanded distal tip 104 has a cross-sectional diameter measured with respect to the inner wall 106 that is greater than a cross-sectional diameter of the intermediate portion of the shaft 103 measured with respect to the inner wall 106. As also shown in FIG. 1, the expanded distal tip 104 has a cross-sectional diameter measured with respect to the outer wall 108 that is greater than a cross-sectional diameter of the intermediate portion of the shaft 103 measured with respect to the outer wall 108. FIGS. 1, 2 and 5 also show that the expanded distal tip 104 may include one or more tip apertures 114 running radially outward from the inner wall 106 of the shaft 102 to the outer wall 108 of the shaft, which may be used to receive various diagnostic instruments and/or for aspirating embolic debris. The distal tip 104 may optionally include a soft and substantially flexible atraumatic material 116 near the distal end 107 of the distal tip 104. The atraumatic material 116 may be made out of any suitable material, such as polyethyltetrafluride (PET), polytetraflurethylene (PTFE), polyamide, or any other polyester compounds. This atraumatic portion 116 of the distal tip 104 may optionally be coated or constructed with a material of higher atomic density to aid in visualizing the distal tip 104, for instance, under fluoroscopy.

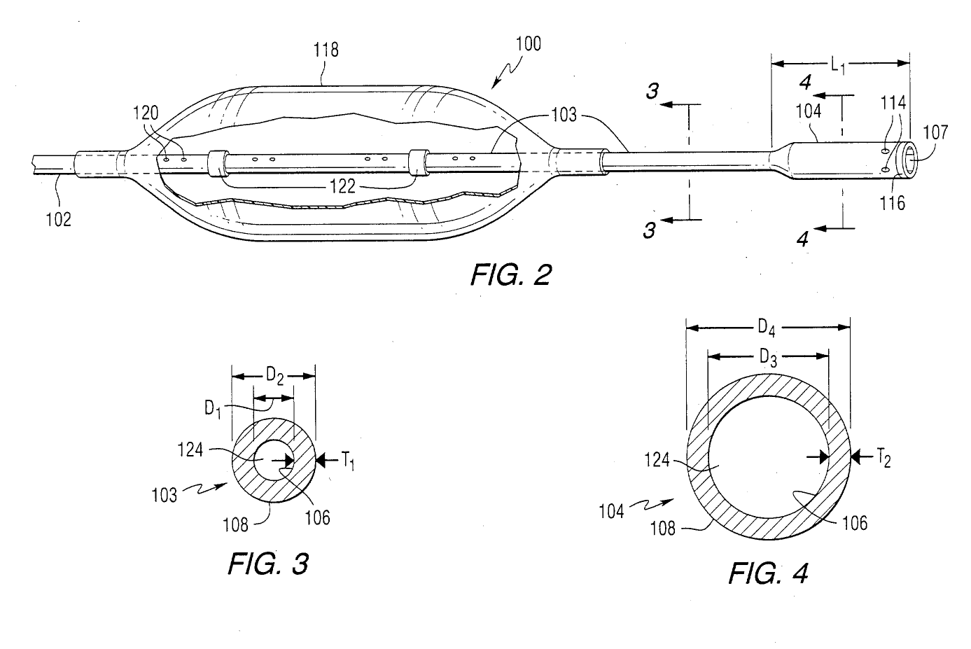

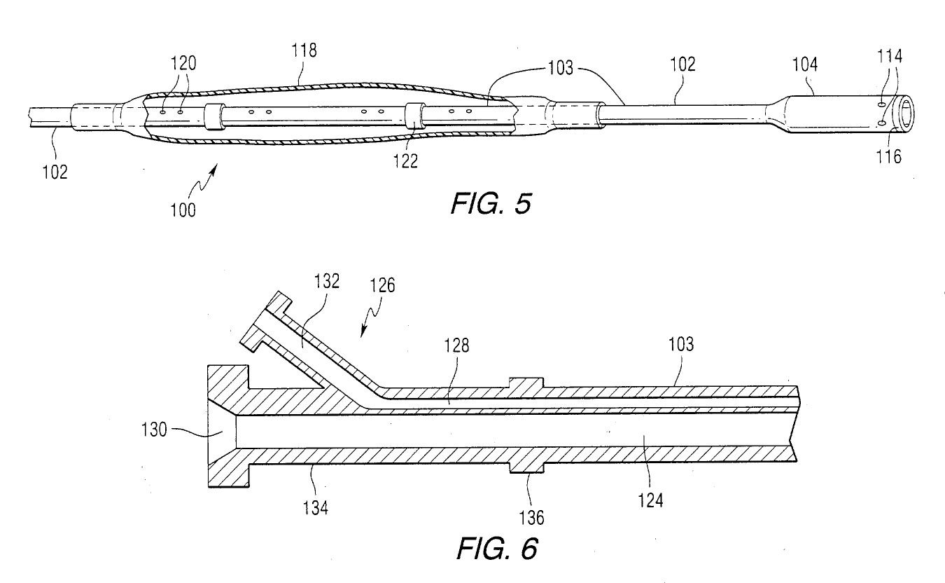

FIGS. 1, 2 and 5 show that the catheter apparatus 100 may include a radially expandable segment, such as an inflatable balloon segment 118, disposed on the intermediate portion 103 of the shaft 102. The inflatable balloon segment 118 may be made out of any suitable material, such as but not limited to, PET, polyethylene, polyamide, PTFE, or other suitable materials that can exert a sufficient radial force to expand a stent or dilate a stenotic portion of an artery. FIGS. 1 and 2 show the inflatable balloon segment 118 in a substantially inflated position and FIG. 5 shows the inflatable balloon segment 118 in a substantially deflated position. FIGS. 1, 2 and 5 also show that the intermediate portion 103 of the shaft 102 containing the inflatable balloon segment 118 may include one or more shaft apertures 120 to allow for the inflatable balloon segment 118 to be inflated and/or deflated. The intermediate portion 103 containing inflatable balloon segment 118 may also include one or more radiopaque markers 122 constructed with a material of higher atomic density to help show the location of the inflatable balloon segment 118 on the shaft 102. The inflatable balloon segment 118 may be used to provide traditional balloon angioplasty to a blood vessel narrowed by stenosis, however, the inflatable balloon segment may also be lightly inflated or deflated to help align the expanded distal tip 104 of the catheter apparatus 100 with a thromboembolic protection device when such a device is being retrieved. Such an alignment may be needed if a blood vessel is tortuous, preventing the catheter apparatus 100 from naturally aligning with a thromboembolic protection device.

As most clearly shown in FIGS. 1 and 6, the shaft 102 may include an interior cavity defining a first lumen 124 running inside the catheter 100 substantially from a proximal end 126 of the catheter 100, as shown in FIG. 6, and extending substantially to the expanded distal tip 104 of the catheter 100 as shown in FIG. 1. The first lumen 124 may be used to accommodate guide wires and/or other diagnostic devices or instruments. As also shown in FIGS. 1 and 6, the shaft 102 also may include an interior cavity defining a second lumen 128 running adjacent to the first lumen 124 substantially from the proximal end 126 of the catheter 100, as shown in FIG. 6, and extending substantially to the intermediate portion 103 of the shaft 102 containing the inflatable balloon segment 118, as shown in FIG. 1. This second lumen 128 may be used, for example, to provide gases, liquids, and/or other materials via the shaft apertures 120 to the inflatable balloon segment 118 for the purposes of inflating or deflating the balloon segment.

FIG. 3 is a cross-sectional view of the intermediate portion 103 of the shaft 102 of the catheter apparatus 100 shown in FIG. 2 taken along the line 3-3. The cross section of the catheter 100 shown in FIG. 3 may have an inner diameter D₁ defined and measured with respect to the inner wall 106 of the shaft 102, an outer diameter D₂ defined and measured with respect to the outer wall 108 of the shaft 102, and a thickness T₁ defined as the distance between the inner wall 106 and the outer wall 108 of the shaft 102. The inner diameter D₁ may range from about 0.4 mm to about 0.6 mm, preferably from about 0.45 mm to about 0.55 mm. A particularly preferred diameter D₁ may be about 0.48 mm. The outer diameter D₂ may range from about 0.9 mm to about 3 mm, preferably from about 1.5 mm to about 3 mm. A particularly preferred diameter D₂ may be about 2.5 mm. The thickness T₁ may range from about 0.25 mm to about 1.2 mm, preferably from about 0.5 mm to about 1.2 mm. A particularly preferred thickness T₁ may be about 1 mm. Although a particular cross-sectional piece of the intermediate portion 103 of the catheter shaft 102 is shown in FIG. 3, it is to be understood that the diameters D₁ and D₂, and the thickness T₁ may be measured at other locations along the intermediate portion of the catheter shaft, such as the portion of the shaft 102 containing the inflatable balloon segment 118. The first lumen 124 and the second lumen 128 are both illustrated in the cross section of the catheter 100 shown in FIG. 3.

FIG. 4 shows a cross-sectional portion of the expanded distal tip 104 of the catheter 100 shown in FIG. 2 taken along the line 4-4. As shown in FIG. 4, an inner diameter D₃ may be defined and measured with respect to the inner wall 106 of the shaft 102, an outer diameter D₄ may be defined and measured with respect to the outer wall 108 of the shaft 102, and a thickness T₂ may be defined as the distance between the inner wall 106 and the outer wall 108 of the shaft 102. The inner diameter D₃ may range from about 0.8 mm to about 1.2 mm, preferably from about 0.95 mm to about 1.1 mm. A particularly preferred diameter D₃ may be about 1 mm. The outer diameter D₄ may range from about 1.3 mm to about 3.6 mm, preferably from about 2 mm to about 3.3 mm. A particularly preferred diameter D₄ may be about 3 mm. The thickness T₂ may range from about 0.25 mm to about 1.2 mm, preferably from about 0.5 mm to about 1.1 mm. A particularly preferred thickness T₂ may be about 1.0 mm. The first lumen 124 is also shown in FIG. 4. FIGS. 3 and 4 illustrate that the inner cross-sectional diameter D₃ of the expanded distal tip 104 is greater than the inner cross sectional diameter D₁ of the intermediate portion 103 of the shaft 102, and that the outer cross-sectional diameter D₄ of the expanded distal tip 104 is greater than the outer cross-sectional diameter D₂ of the intermediate portion 103 of the shaft 102. FIGS. 3 and 4 also illustrate that the thickness T₂ is substantially equal to the thickness T₁.

In this embodiment, a ratio of the diameter D₃ to D₁ may be defined as D₃:D₁. D₃:D₁ may range from about 1.6:1 to about 2.4:1, preferably from about 1.8:1 to about 2.2:1. In a particularly preferred embodiment, D₃:D₁ may be about 2:1. In this embodiment, a ratio of the diameter D₄ to the diameter D₂ may also be defined as D₄:D₂. D₄:D₂ may range from about 1.1:1 to about 1.4:1, preferably from about 1.1:1 to about 1.3:1. In a particularly preferred embodiment, D₄:D₂ may be about 1.2:1.

FIG. 2 shows that the length of the expanded distal tip 104 may be defined as L₁. The length L₁ may range from about 0.3 cm to about 1 cm, preferably from about 0.5 cm to about 0.7 cm. A particularly preferred length L₁ may be about 0.7 cm. In this embodiment, a ratio of the diameter D₃ of the expanded distal tip 104 to the length L₁ of the expanded distal tip 104 may be defined as D₃:L₁. D₃:L₁ may range from about 0.27:1 to about 0.12:1, preferably from about 0.19:1 to about 0.13:1. In a particular embodiment, D₃:L₁ may be about 0.14:1. In this embodiment, a ratio of the diameter D₄ of the expanded distal tip 104 to the length L₁ of the distal tip 104 may be defined as D₄:L₁. D₄:L₁ may range from about 0.44:1 to about 0.36:1, preferably from about 0.4:1 to about 0.39:1. In a particular embodiment of the invention, D₄:L₁ may be about 0.4:1.

FIG. 6 shows a proximal end 126 of the catheter apparatus 100. The proximal end 126 of the catheter 100 includes a first port 130 in flow communication with the first lumen 124, and a second port 132 in flow communication with the second lumen 128. The first port 130 and the second port 132 may both be substantially enclosed in a Y-shaped housing 134 as illustrated in FIG. 6. FIG. 6 shows that the Y-shaped housing 134 may also include a reinforcing lip or ridge 136 for providing the Y-shaped housing 134 with added structural support. As shown in FIG. 6, the Y-shaped housing 134 may be attached to the shaft 102 with any suitable fastening means, or optionally may be formed as an integral part of the catheter 100 during manufacture. The first port 130 may be used to supply the first lumen 124 with guide wires, suction for aspirating embolic material, and/or other diagnostic instruments, and the second port 132 may be used to supply the second lumen 128 with materials for inflating and deflating the inflatable balloon segment 118, such as, but not limited to, various gases and liquids.

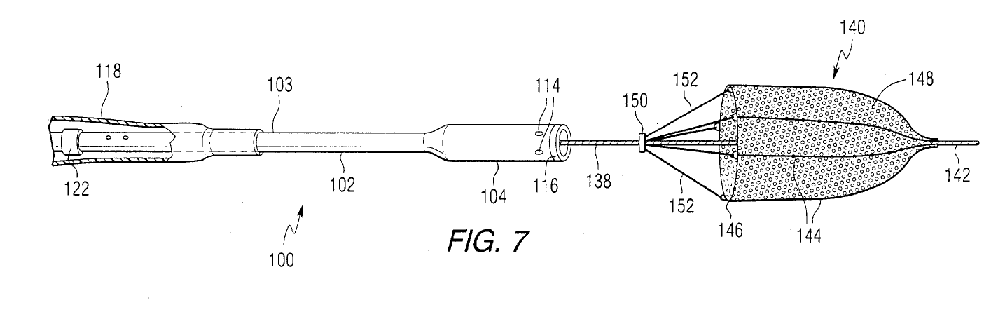

FIGS. 7-9 show the catheter apparatus 100 in conjunction with a guide wire 138 and a thromboembolic protection device, such as an embolic filter assembly 140, mounted substantially near a distal end 142 of the guide wire 138. The guide wire 138 may be made of any suitable material, such as stainless steel, nickel titanium alloy (Nitinol), coiled spring stainless steel or other related alloys, and the first lumen 124 of the catheter apparatus 100 may be structured and arranged to receive the guide wire 138 within the first lumen 124. In one embodiment, the guide wire 138 may run substantially along the entire length of the first lumen 124, and a proximal end (not shown) of the guide wire 138 may protrude from the first port 130 of the first lumen 124. Although a guide wire is shown in this embodiment, other types of flexible tubing may also be used. The tubing or guide wire preferably has an outer diameter of greater than about 0.05 cm and less than 0.25 cm, however guide wires with other suitable diameters may be used. For example, the guide wire 138 may have an outer diameter of about 0.09 cm.

The embolic filter assembly 140 may be of any suitable construction for collecting and containing embolic material that is well known in the art. In one embodiment, as most clearly illustrated in FIG. 7, the embolic filter assembly 140 may include a plurality of ribs 144 spaced around the external circumference of the guide wire 138. More or less ribs may be used. For example, although four ribs are shown, a device with six ribs may be constructed, and the ribs may be spaced at various intervals around the circumference of the guide wire 138, for example, in an equiangular fashion. The ribs 144 are preferably formed of a resilient material, such as stainless steel, or Nitinol memory metal or plastic, which is pre-stressed or pre-formed resulting in an expandable or outward bias. The tips 146 of the ribs 144 may be preferably curved inward to minimize trauma to the blood vessel wall.

A filter material 148 spans the gaps between and is secured to the ribs 144. The filter material 148 is preferably a finely porous mesh capable of trapping embolic material broken loose from interventional procedures, but coarse enough to allow blood to pass through. Suitable filter materials include porous PTFE, fabrics and metals. When metal such as Nitinol memory metal is used as the filter material, it preferably has a low profile and facilitates trackability of the filter during use. The filter material 148 may be attached to the ribs 144 by any suitable means such as sutures, pockets, adhesives and the like. In one embodiment, the filter material 148 may be tied to the ribs 144 by sutures which also may act as control strings of the embolic filter assembly 140.

In many medium sized blood vessels, the embolic filter assembly 140 may expand to a diameter against the wall of the vessel from about 4 mm to about 10 mm, often from about 6 mm to about 8 mm. In larger vessels such as the aorta, the embolic filter assembly 140 may expand to a diameter from about 10 mm to about 30 mm, often from about 12 mm to about 20 mm.

As most clearly illustrated in FIG. 7, the tips 146 of the ribs 144 may be attached to a collar 150 via a plurality of control strings 152. The control strings 152 may be made of any suitable material such as metal wires, sutures or suture-like materials. The diameter of each control string 152 is preferably 0.03 cm or less. The collar 150 is preferably in sliding engagement with the guide wire 138, so that the collar may move freely along the guide wire. In another embodiment, a collar is not used, and instead the control strings 152 may be attached directly to the guide wire 138 by any suitable means.

In one embodiment, the embolic filter assembly 140 may be introduced into a blood vessel with an introducer sheath (not shown). In this embodiment, the introducer sheath may encase the embolic filter assembly 140, keeping the embolic filter assembly in a substantially closed position. Once the embolic filter assembly 140 has been placed in a blood vessel at an appropriate location, the introducer sheath may be removed from the embolic filter assembly, thereby allowing the resilient ribs 144 to naturally expand, in turn causing the embolic filter assembly 140 to open to a substantially expanded position, as shown in FIG. 7. In one embodiment, the introducer sheath may be “peeled” away from the embolic filter assembly 140 and removed from the patient using a string, cord, suture, or other appropriate peeling means. In another embodiment, the guide wire may be held in a substantially stationary position, and the introducer sheath may be slideably removed from the embolic filter assembly 140 and subsequently removed from the patient.

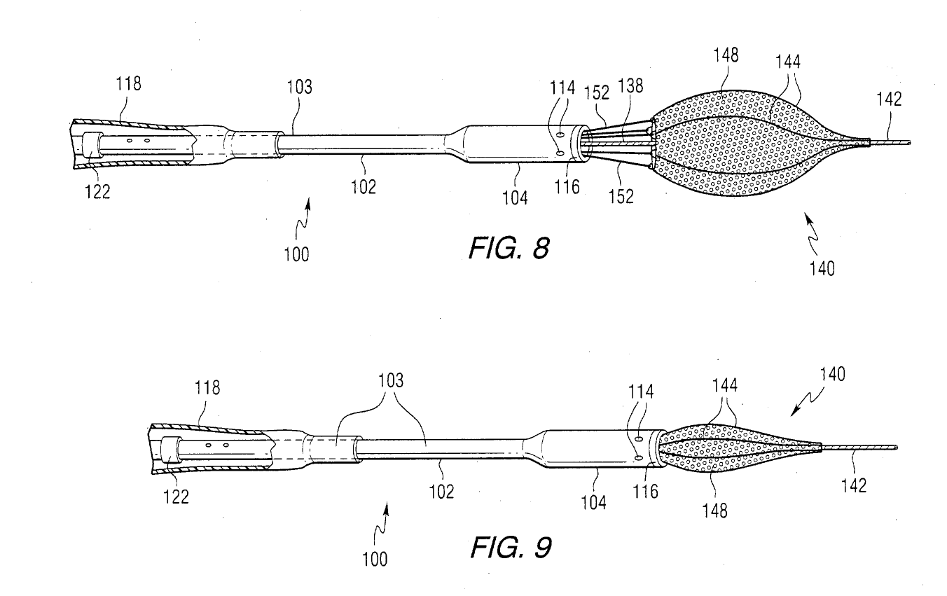

FIGS. 8 and 9 illustrate how the catheter apparatus 100 may be used to retract the embolic filter assembly 140. Once the interventional procedure is complete, such as a balloon angioplasty procedure, and the embolic filter assembly 140 has captured any loose or dislodged embolic material, the catheter 100 may be advanced towards the distal end 142 of the guide wire 138 until the distal tip 104 of the catheter 100 meets the control strings 152 of the embolic filter assembly 140. As shown in FIG. 8, the catheter apparatus 100 may then continue to be advanced towards the distal end 142 of the guide wire 138, and the operator may pull on a proximal end (not shown) of the guide wire 138 which may protrude from the first port 130 of the first lumen 124, which will preferably cause the collar 150 and the control strings 152 to be pulled into the distal tip 104 of the catheter 100, thereby causing the embolic filter assembly 140 to begin to collapse. As shown in FIG. 9, the guide wire 138 may continue to be pulled until the embolic filter assembly 140 and the captured embolic material (not shown) are safely retracted and stored within the distal tip 104 of the catheter apparatus 100. As shown in FIG. 9, only a portion of the embolic filter assembly 140 need be stored in the distal tip 104. In another embodiment, the guide wire 138 may be pulled until the entire embolic filter assembly 140 is stored within the distal tip 140. In another embodiment, the guide wire 138 may remain substantially stationary, and the catheter apparatus 100 may be advanced towards the distal end 142 of the guide wire 138 until a portion of the embolic filter assembly 140 or the entire embolic filter assembly 140 is safely stored within the distal tip 104 of the catheter apparatus 100.

In another embodiment of the invention, an embolic filter assembly and guide wire combination may be used with the present invention as disclosed in copending commonly owned U.S. patent application Ser. No. 09/476,829 filed Jan. 3, 2000, which is hereby incorporated by reference. In this embodiment, an embolic filter assembly may be substantially structured and arranged as described above, however, multiple control strings may be attached to an actuator located near a proximal end of a guide wire. The control strings may run inside the guide wire and may exit the guide wire through holes located in a collar, such as the collar 150 described above. The control strings may then be secured to the tips of a plurality of ribs of the embolic filter assembly. To open the embolic filter assembly, the actuator may be pushed forward, releasing tension upon the control strings and allowing the embolic filter assembly to self-expand. When the interventional procedure is complete, the actuator may be pulled, tensioning the control strings and causing the embolic filter assembly to retract, allowing the dislodged embolic material to be retained in a deep pocket formed by the filter material of the embolic filter assembly. The catheter apparatus 100 may then be advanced toward a distal end of the guide wire until the collapsed embolic filter assembly is safely stored within the distal tip 104 of the catheter apparatus 100, or the guide wire 138 may be pulled until the collapsed embolic filter assembly is safely stored within the distal tip 104 of the catheter apparatus 100.

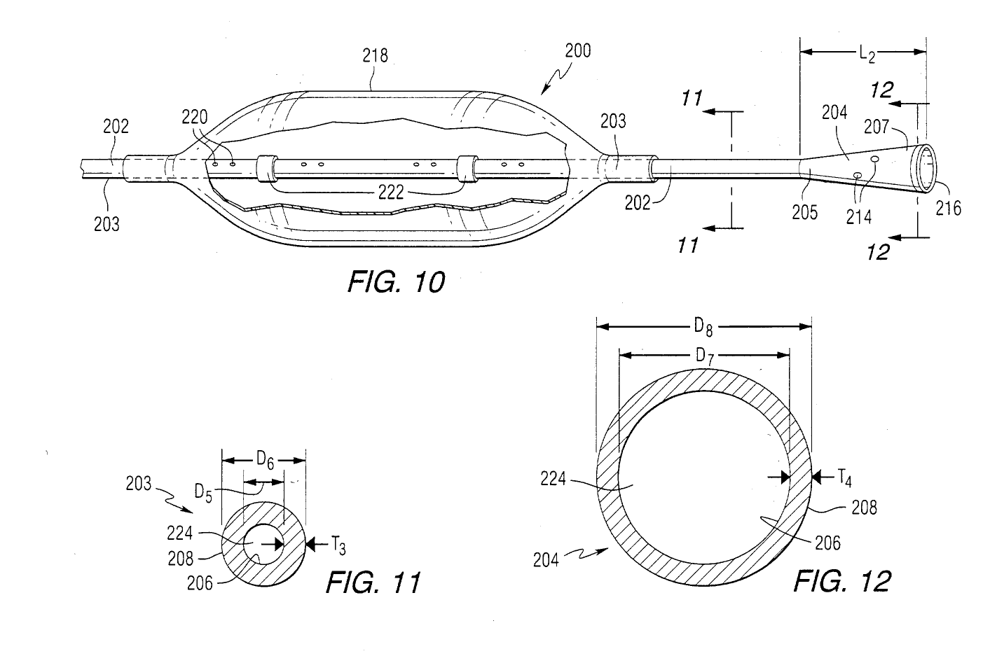

FIGS. 10-12 show a catheter apparatus 200 in accordance with another embodiment of the present invention. The catheter 200 includes a shaft 202, and the shaft includes an intermediate portion 203 and an expanded distal tip 204. In this embodiment the distal tip may be substantially conical shaped, as shown in FIG. 10, with the diameter of the distal tip 204 gradually increasing towards the distal end 207 of the distal tip 204. FIG. 10 shows that the distal tip 204 may include one or more tip apertures 214 running radially outward from an inner wall 206 of the shaft to an outer wall 208 of the shaft, which may be used for diagnostic purposes such as aspirating or removing thromboembolic material or other particles from a blood vessel. The distal tip 204 may optionally include a soft and substantially flexible atraumatic material 216 near the distal end 207 of the distal tip 204. This atraumatic material 216 may optionally be coated or constructed with a material of higher atomic density to aid in visualizing the distal tip 204, for instance, under fluoroscopy.

FIG. 10 shows that the catheter apparatus 200 may include a radially expandable segment, such as an inflatable balloon segment 218, disposed on the intermediate portion 203 of the shaft 202. FIG. 10 also shows that the intermediate portion 203 of the shaft 202 containing the inflatable balloon segment 218 may include one or more shaft apertures 220 to allow for the inflatable balloon segment 218 to be inflated and/or deflated. The intermediate portion 203 of the shaft 202 containing inflatable balloon segment 218 may also include one or more radiopaque markers 222 constructed with a material of higher atomic density to help show the location of the inflatable balloon segment 218 on the shaft 202.

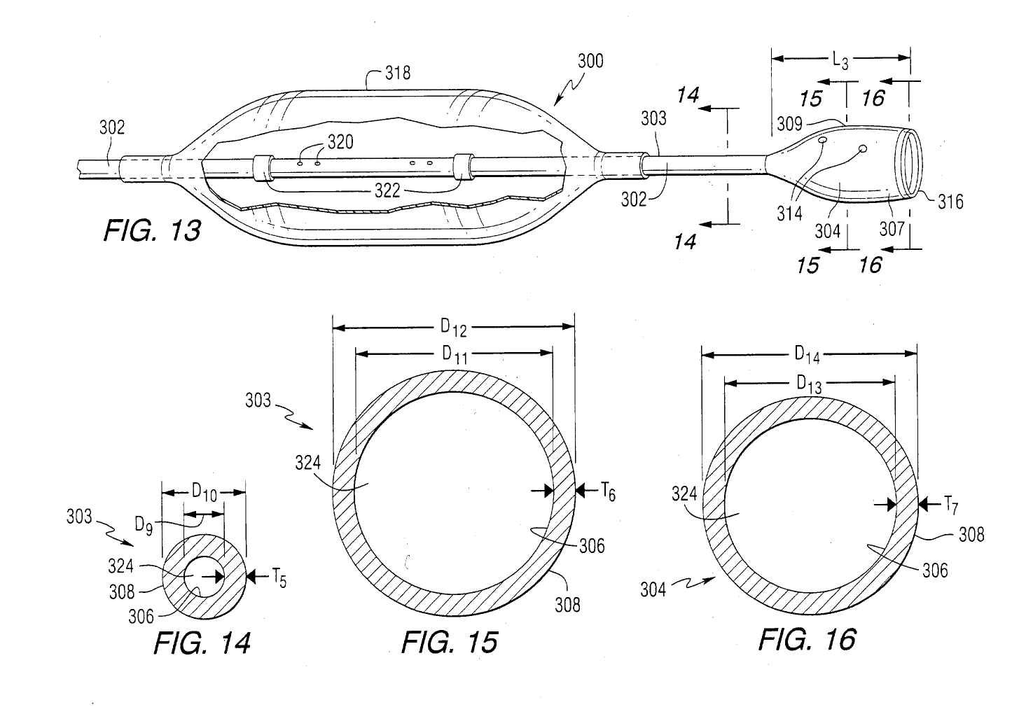

The shaft 202 also includes an interior cavity defining a first lumen 224, of which a cross-sectional portion is shown in FIGS. 13 and 14, running inside the catheter substantially from a proximal end (not shown) of the catheter and extending substantially to the distal tip 204 of the catheter 200. The first lumen 224 may be used to accommodate guide wires and/or other diagnostic devices or instruments. The shaft 202 also may include an interior cavity defining a second lumen 228 running adjacent to the first lumen 224 substantially from the proximal end (not shown) of the catheter 200 and extending substantially to the intermediate portion 203 of the shaft 202 containing the inflatable balloon segment 218. This second lumen 228 may be used, for example, to provide gases, liquids, or other materials via the shaft apertures 220 to the inflatable balloon segment 218 for the purposes of inflating or deflating the balloon segment.

FIG. 11 is a cross-sectional view of the intermediate portion 203 of the shaft 202 of the catheter apparatus 200 shown in FIG. 10 taken along the line 11-11. FIG. 11 shows that the shaft 202 includes inner wall 206 and outer wall 208. The cross section of the catheter 200 shown in FIG. 11 may have an inner diameter D₅ defined and measured with respect to the inner wall 206 of the shaft 202, an outer diameter D₆ defined and measured with respect to the outer wall 208 of the shaft 202, and a thickness T₃ defined as the distance between the inner wall 206 and the outer wall 208 of the shaft 202. The inner diameter D₅ may range from about 0.4 mm to about 0.6 mm, preferably from about 0.45 mm to about 0.55 mm. A particularly preferred inner diameter D₅ may be about 0.48 mm. The outer diameter D₆ may range from about 0.9 mm to about 3 mm, preferably from about 1.5 mm to about 3 mm. A particularly preferred outer diameter D₆ may be about 2.5 mm. The thickness T₃ may range from about 0.25 mm to about 1.2 mm, preferably from about 0.5 mm to about 1.2 mm.

A particularly preferred thickness T₃ may be about 1 mm. Although a particular cross-sectional piece of the intermediate portion 203 of the catheter shaft 202 is shown in FIG. 11, it is to be understood that the diameters D₅ and D₆, and the thickness T₃ may be measured at other locations along the intermediate portion of the catheter shaft, such as the intermediate portion 203 of the shaft 202 containing the inflatable balloon segment 218. The first lumen 224 is illustrated in the cross section of the shaft 202 shown in FIG. 11.

FIG. 12 shows a cross-sectional portion of the expanded distal tip 204 of the catheter 200 shown in FIG. 10 taken along the line 12-12. An inner diameter D₇ may be defined and measured with respect to the inner wall 206 of the shaft 202, an outer diameter D₈ may be defined and measured with respect to the outer wall 208 of the shaft 202, and a thickness T₄ may be defined as the distance between the inner wall 206 and the outer wall 208 of the shaft 202. The inner diameter D₇ may range from about 0.8 mm to about 1.2 mm, preferably from about 0.95 mm to about 1.1 mm. A particularly preferred diameter D₇ may be about 1 mm. The outer diameter D₈ may range from about 1.3 mm to about 3.6 mm, preferably from about 2 mm to about 3.3 mm. A particularly preferred diameter D₈ may be about 3 mm. The thickness T₄ may range from about 0.25 mm to about 1.2 mm, preferably from about 0.5 mm to about 1.1 mm. A particularly preferred thickness T₄ may be about 1 mm. The first lumen 224 is also shown in FIG. 12.

FIGS. 11 and 12 illustrate that the inner cross-sectional diameter D₇ of the expanded distal tip 204 is greater than the inner cross-sectional diameter D₅ of the intermediate portion 203 of the shaft 202, and that the outer cross-sectional diameter D₈ of the expanded distal tip 204 is greater than the outer cross-sectional diameter D₆ of the intermediate portion 203 of the shaft 202. FIGS. 11 and 12 also illustrate that the thickness T₄ is substantially equal to the thickness T₃.

In this embodiment, a ratio of the diameter D₇ to D₅ may be defined as D₇:D₅. D₇:D₅ may range from about 1.6:1 to about 2.4:1, preferably from about 1.8:1 to about 2.2:1. In a particularly preferred embodiment, D₇:D₅ may be about 2:1. In this embodiment, a ratio of the diameter D₈ to the diameter D₆ may also be defined as D₈:D₆. D₈:D₆ may range from about 1.1:1 to about 1.4:1, preferably from about 1.1:1 to about 1.3:1. In a particularly preferred embodiment, D₈:D₆ may be about 1.2:1.

FIG. 10 shows that the length of the expanded distal tip 204 may be defined as L₂. The length L₂ may range from about 0.3 cm to about 1 cm, preferably from about 0.5 cm to about 0.7 cm. A particularly preferred length L₂ may be about 0.7 cm. In this embodiment, a ratio of the diameter D₇ of the distal tip 204 to the length L₂ of the distal tip 204 may be defined as D₇:L₂. D₇:L₂ may range from about 0.27:1 to about 0.12:1, preferably from about 0.19:1 to about 0.13:1. In a particular embodiment, D₇:L₂ may be about 0.14:1. In this embodiment, a ratio of the diameter D₈ of the distal tip 204 to the length L₂ of the distal tip 204 may be defined as D₈:L₂. D₈:L₂ may range from about 0.44:1 to about 0.36:1, preferably from about 0.4:1 to about 0.39:1. In a particular embodiment of the invention, D₈:L₂ maybe about 0.4:1.

FIGS. 13-16 show a catheter apparatus 300 in accordance with another embodiment of the present invention. The catheter apparatus 300 includes a shaft 302, and the shaft includes an intermediate portion 303 and an expanded distal tip 304. In this embodiment the distal tip 304 is substantially bulb-shaped. As used herein, the term “bulb-shaped” refers to an expanded distal tip having at least a portion of the inner and/or outer walls curved. FIG. 13 shows that the expanded distal tip 304 may include one or more tip apertures 314 running radially outward from an inner wall 306 of the shaft to an outer wall 308 of the shaft, which may be used for diagnostic purposes such as aspirating or removing thromboembolic material or other particles from a blood vessel. The expanded distal tip 304 may optionally include a soft and substantially flexible atraumatic material 316 at the distal end 307 of the distal tip 304. This atraumatic material 316 may optionally be coated or constructed with a material of higher atomic density to aid in visualizing the distal tip 304, for instance, under fluoroscopy.

FIG. 13 shows that the catheter 300 may include a radially expandable segment, such as an inflatable balloon segment 318, disposed on the intermediate portion 303 of the shaft 302. FIG. 13 also shows that the intermediate portion 303 of the shaft 302 containing the inflatable balloon segment 318 may include one or more shaft apertures 320 to allow for the inflatable balloon segment 318 to be inflated and/or deflated. The intermediate portion 303 of the shaft 302 containing inflatable balloon segment 318 may also include one or more radiopaque markers 322 constructed with a material of higher atomic density to help show the location of the inflatable balloon segment 318 on the shaft 302.

The catheter shaft 302 also preferably includes an interior cavity defining a first lumen 324, of which a cross-sectional portion is shown in FIGS. 14-16, running inside the catheter 300 substantially from a proximal end (not shown) of the catheter 300 and extending substantially to the expanded distal tip 304 of the catheter 300. The first lumen 324 may be used to accommodate guide wires and/or other diagnostic devices or instruments. The catheter shaft 302 also may include an interior cavity defining a second lumen 328 running adjacent to the first lumen 324 substantially from the proximal end (not shown) of the catheter 300 and extending substantially to the intermediate portion 303 of the shaft 302 containing the inflatable balloon segment 318. This second lumen 328 may be used, for example, to provide gases, liquids, or other materials via the shaft apertures 320 to the inflatable balloon segment 318 for a purpose such as inflating and/or deflating the balloon segment.

FIG. 14 is a cross-sectional view of the intermediate portion 303 of the shaft 302 of the catheter 300 shown in FIG. 13 taken along the line 14-14. FIG. 14 shows that the shaft 302 includes inner wall 306 and outer wall 308. The cross section of the catheter 300 shown in FIG. 14 may have an inner diameter D₉ defined and measured with respect to the inner wall 306 of the shaft 302, an outer diameter D₁₀ defined and measured with respect to the outer wall 308 of the shaft 302, and a thickness T₅ defined as the distance between the inner wall 306 and the outer wall 308 of the shaft 302. The inner diameter D₉ may range from about 0.4 mm to about 0.6 mm, preferably from about 0.45 mm to about 0.55 mm. A particularly preferred diameter D₉ may be about 0.48 mm. The outer diameter D₁₀ may range from about 0.9 mm to about 3 mm, preferably from about 1.4 mm to about 3 mm. A particularly preferred diameter D₁₀ may be about 2.5 mm. The thickness T₅ may range from about 0.25 mm to about 1.2 mm, preferably from about 0.5 mm to about 1.2 mm. A particularly preferred thickness T₅ may be about 1 mm. Although a particular cross-sectional piece of the intermediate portion 303 of the catheter shaft 302 is shown in FIG. 14, it is to be understood that the diameters D₉ and D₁₀, and the thickness T₅ may be measured at other locations along the intermediate portion of the catheter shaft, such as the intermediate portion 303 of the shaft 302 containing the inflatable balloon segment 318. The first lumen 324 is illustrated in the cross section of the shaft 302 shown in FIG. 14.

FIG. 15 illustrates a cross-sectional portion of the expanded distal tip 304 of the shaft 302 of the catheter apparatus 300 shown in FIG. 13 taken along the line 15-15, which is at the approximate midpoint 309 of the length of the expanded distal tip 304. In this embodiment, the cross-sectional diameter of the distal tip 304, measured with respect to the inner wall 306 and outer wall 308 of the shaft, gradually increases from a proximal end 305 of the expanded distal tip to approximately the midpoint 309 of the expanded distal tip, and then gradually decreases slightly from approximately the midpoint 309 of the expanded distal tip to the distal end 307 of the expanded distal tip 304. FIG. 15 shows that an inner diameter D₁₁ may be defined and measured with respect to the inner wall 306 of the shaft 302, an outer diameter D₁₂ may be defined and measured with respect to an outer wall 308 of the shaft 302, and a thickness T₆ may be defined as the distance between the inner wall 306 and the outer wall 308 of the shaft 302. The inner diameter D₁₁ may range from about 1 mm to about 1.5 mm, preferably from about 1.15 mm to about 1.3 mm. A particularly preferred diameter D₁₁ may be about 1.2 mm. The outer diameter D₁₂ may range from about 1.5 mm to about 3.8 mm, preferably from about 2.2 mm to about 3.5 mm. A particularly preferred diameter D₁₂ may be about 3.2 mm. The thickness T₆ may range from about 0.25 mm to about 1.2 mm, preferably from about 0.5 mm to about 1.1 mm. A particularly preferred thickness T₆ may be about 1.0 mm. The first lumen 324 is also illustrated in FIG. 15.

FIG. 16 shows a cross-sectional portion of the expanded distal tip 304 of the catheter 300 shown in FIG. 13 taken along the line 16-16, which is approximately at the distal end 307 of the expanded distal tip 304. As shown in FIG. 16, an inner diameter D₁₃ may be defined and measured with respect to the inner wall 306 of the shaft 302, an outer diameter D₁₄ may be defined and measured with respect to the outer wall 308 of the shaft 302, and a thickness T₇ may be defined as the distance between the inner wall 306 and the outer wall 308 of the shaft 302. The inner diameter D₁₃ may range from about 0.8 mm to about 1.2 mm, preferably from about 0.95 mm to about 1.1 mm. A particularly preferred diameter D₁₃ may be about 1 mm. The outer diameter D₁₄ may range from about 1.3 mm to about 3.6 mm, preferably from about 2 mm to about 3.3 mm. A particularly preferred diameter D₁₄ may be about 3 mm. The thickness T₇ may range from about 0.25 mm to about 1.2 mm, preferably from about 0.5 mm to about 1.1 mm. A particularly preferred thickness T₇ may be about 1.0 mm. The first lumen 324 is also shown in FIG. 16.

FIGS. 14 and 15 illustrate that the diameter D₁₁ of the expanded distal tip 304 is greater than the diameter D₉ of the intermediate portion 403 of the shaft 302, and that the diameter D₁₂ of the expanded distal tip 304 is greater than the diameter D₁₀ of the shaft 302. FIGS. 14 and 15 also illustrate that the thickness T₅ is substantially equal to the thickness T₆.

FIGS. 14 and 16 illustrate that the diameter D₁₃ of the expanded distal tip 304 is greater than the diameter D₉ of the shaft 302, and that the diameter D₁₄ of the expanded distal tip 304 is greater than the diameter D₁₀ of the shaft 302. FIGS. 14 and 16 also illustrate that the thickness T₅ is substantially equal to the thickness T₇.

In this embodiment, a ratio of the diameter D₁₁ to D₉ may be defined as D₁₁:D₉. D₁₁:D₉ may range from about 2.3:1 to about 2.5:1, preferably from about 2.4:1 to about 2.5:1. In a particularly preferred embodiment, D₁₁:D₉ may be about 2.5:1. In this embodiment, a ratio of the diameter D₁₂ to the diameter D₁₀ may also be defined as D₁₂:D₁₀. D₁₂:D₁₀ may range from about 1.3:1 to about 1.7:1, preferably from about 1.3:1 to about 1.6:1. In a particularly preferred embodiment, D₁₂:D₁₀ may be about 1.2:1.

FIG. 13 shows that the length of the expanded distal tip 304 may be defined as L₃. The length L₃ may range from about 0.3 cm to about 1 cm, preferably from about 0.5 cm to about 0.7 cm. A particularly preferred length L₃ may be about 0.7 cm. In this embodiment, a ratio of the diameter D₁₁ of the distal tip 304 to the length L₃ of the distal tip 304 maybe defined as D₁₁:L₃. D₁₁:L₃ may range from about 0.38:1 to about 0.14:1, preferably from about 0.23:1 to about 0.19:1. In a particular embodiment, D₁₁:L₃ maybe about 0.17:1. In this embodiment, a ratio of the diameter D₁₂ of the distal tip 304 to the length L₃ of the distal tip 304 may be defined as D₁₂:L₃. D₁₂:L₃ may range from about 0.5:1 to about 0.38:1, preferably from about 0.44:1 to about 0.5:1. In a particular embodiment of the invention, D₁₂:L₃ may be about 0.46:1.

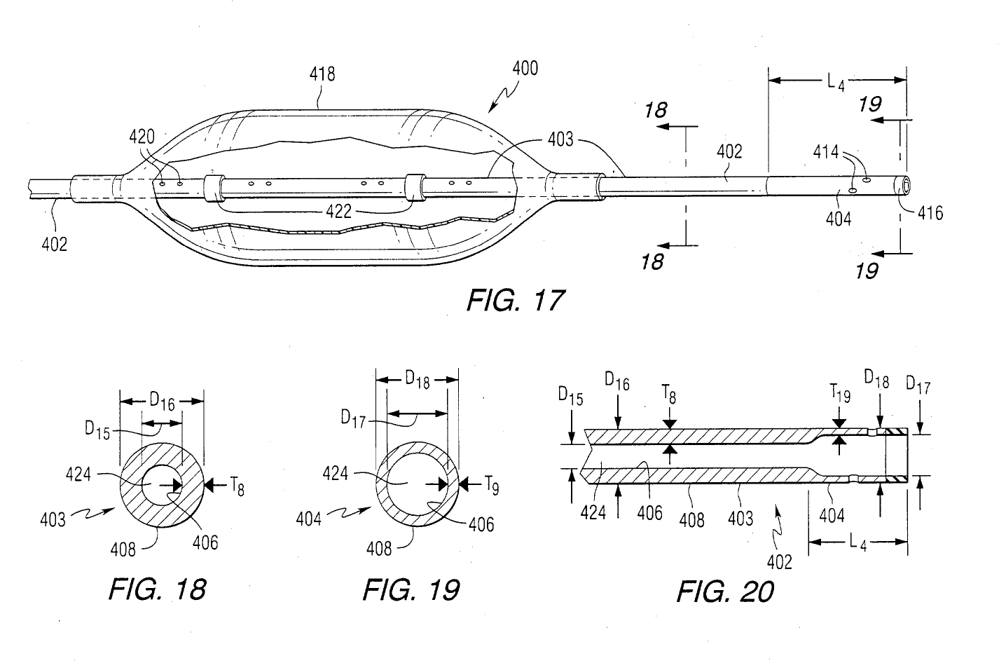

FIGS. 17-20 show a catheter apparatus 400 in accordance with another embodiment of the present invention. The catheter apparatus 400 includes a shaft 402, and the shaft includes an intermediate portion 403 and an expanded distal tip 404. In this embodiment the expanded distal tip 404 may have an outer diameter that is substantially equal to an outer diameter of the intermediate portion of the shaft 403, as shown in FIGS. 17 and 20. FIGS. 17 and 20 illustrate that the expanded distal tip 404 may include one or more tip apertures 414 running radially outward from an inner wall 406 of the shaft to an outer wall 408 of the shaft, which may be used for diagnostic purposes such as aspirating or removing thromboembolic material or other particles from a blood vessel. The expanded distal tip 404 may optionally include a soft and substantially flexible atraumatic material 416 at the distal end 407 of the distal tip 404. This atraumatic material 416 may optionally be coated or constructed with a material of higher atomic density to aid in visualizing the distal tip 404, for instance, under fluoroscopy.

FIG. 17 shows that the catheter 400 may include a radially expandable segment, such as an inflatable balloon segment 418, disposed on the intermediate portion 403 of the shaft 462. FIG. 17 also shows that the intermediate portion 403 of the shaft 402 containing the inflatable balloon segment 418 may include one or more shaft apertures 420 to allow for the inflatable balloon segment 418 to be inflated and/or deflated. The intermediate portion 403 of the shaft 402 containing inflatable balloon segment 418 may also include one or more radiopaque markers 422 constructed with a material of higher atomic density to help show the location of the inflatable balloon segment 418 on the shaft 402.

The catheter shaft 402 also preferably includes an interior cavity defining a first lumen 424, of which a cross-sectional portion is shown in FIGS. 18 and 19, running inside the catheter 400 substantially from a proximal end (not shown) of the catheter 400 and extending substantially to the expanded distal tip 404 of the catheter 400. The first lumen 424 may be used to accommodate guide wires and/or other diagnostic devices or instruments. The catheter shaft 402 also may include an interior cavity defining a second lumen 428 running adjacent to the first lumen 424 substantially from the proximal end (not shown) of the catheter 400 and extending substantially to the intermediate portion 403 of the shaft 402 containing the inflatable balloon segment 418. This second lumen 428 may be used, for example, to provide gases, liquids, or other materials via the shaft apertures 420 to the inflatable balloon segment 418 for the purposes of inflating and/or deflating the balloon segment.

FIG. 18 is a cross-sectional view of the intermediate portion 403 of the shaft 402 of the catheter 400 shown in FIG. 17 taken along the line 18-18. FIG. 18 shows that the shaft 402 includes inner wall 406 and outer wall 408. The cross section of the catheter 400 shown in FIG. 18 may have an inner diameter D₁₅ defined and measured with respect to the inner wall 406 of the shaft 402, an outer diameter D₁₆ defined and measured with respect to the outer wall 408 of the shaft 402, and a thickness T₈ defined as the distance between the inner wall 406 and the outer wall 408 of the shaft 402. The diameter D₁₅ may range from about 0.4 mm to about 0.6 mm, preferably from about 0.45 mm to about 0.55 mm. A particularly preferred diameter D₁₅ may be about 0.48 mm. The diameter D₁₆ may range from about 0.9 mm to about 3 mm, preferably from about 1.5 mm to about 3 mm. A particularly preferred diameter D₁₆ may be about 2.5 mm. The thickness T₈ may range from about 0.25 mm to about 1.2 mm, preferably from about 0.5 mm to about 1.2 mm. A particularly preferred thickness T₈ may be about 1 mm. Although a particular cross-sectional piece of the intermediate portion 403 of the catheter shaft 402 is shown in FIG. 18, it is to be understood that the diameters D₁₅ and D₁₆, and the thickness T₈ may be measured at other locations along the intermediate portion of the catheter shaft, such as the intermediate portion 403 of the shaft 402 containing the inflatable balloon segment 418. The first lumen 424 is illustrated in the cross section of the shaft 402 shown in FIG. 18.

FIG. 19 shows a cross-sectional portion of the expanded distal tip 404 of the shaft 402 of the catheter apparatus 400 shown in FIG. 17 taken along the line 19-19. As shown in FIGS. 19 and 20, an inner diameter D₁₇ may be defined and measured with respect to the inner wall 406 of the shaft 402, an outer diameter D₁₈ may be defined and measured with respect to the outer wall 408 of the shaft 402, and a thickness T₉ may be defined as the distance between the inner wall 406 and the outer wall 408 of the shaft 402. The inner diameter D₁₇ may range from about 0.65 mm to about 1.8 mm, preferably from about 0.95 mm to about 1.65 mm. A particularly preferred diameter D₁₇ may be about 1.48 mm. The outer diameter D₁₈ may range from about 0.9 mm to about 3 mm, preferably from about 1.5 mm to about 3 mm. A particularly preferred diameter D₁₈ may be about 2.5 mm. The thickness T₉ may range from about 0.125 mm to about 0.6 mm, preferably from about 0.25 mm to about 0.55 mm. A particularly preferred thickness T₉ may be about 0.5 mm. The first lumen 424 is also shown in FIG. 19.

FIGS. 18-20 illustrate that the inner diameter D₁₇ of the expanded distal tip 404 is greater than the diameter D₁₅ of the intermediate portion 403 of the shaft 402. However, in this embodiment, the diameter D₁₈ of the expanded distal tip 404 is substantially equal to the diameter D₁₆ of the intermediate portion 403 of the shaft 402. FIGS. 18-20 also illustrate that the thickness T₈ is greater than the thickness T₉.

In this embodiment, a ratio of the diameter D₁₇ to D₁₅ may be defined as D₁₇:D₁₅. D₁₇:D₁₅ may range from about 1.63:1 to about 3:1, preferably from about 2.1:1 to about 3:1. In a particularly preferred embodiment, D₁₇:D₁₅ may be about 3:1. In this embodiment, a ratio of the thickness T₈ to the thickness T₉ may also be defined as T₈:T₉. T₈:T₉ may range from about 1.5:1 to about 2.5:1, preferably from about 1.75:1 to about 2.25:1. In a particularly preferred embodiment, T₈:T₉ may be about 2:1.

FIGS. 17 and 20 show that the length of the distal tip 404 may be defined as L₄. The length L₄ may range from about 0.3 cm to about 1 cm, preferably from about 0.5 cm to about 0.7 cm. A particularly preferred length L₄ may be about 0.7 cm. In this embodiment, a ratio of the diameter D₁₇ of the expanded distal tip 404 to the length L₄ of the expanded distal tip 404 may be defined as D₁₇:L₄. D₁₇:L₄ may range from about 0.22:1 to about 0.18:1, preferably from about 0.22:1 to about 0.19:1. In a particular embodiment, D₁₇:L₄ may be about 0.21:1. In this embodiment, a ratio of the thickness T₉ to the length L₄ of the expanded distal tip 404 may be defined as T₉:L₄. T₉:L₄ may range from about 0.04:1 to about 0.08:1, preferably from about 0.05:1 to about 0.08:1. In a particular embodiment of the invention, T₉:L₄ may be about 0.07:1.

It will be appreciated that catheter apparatus 200, 300 and 400 may all be used in conjunction with a guide wire and embolic filter assembly as disclosed and described herein.

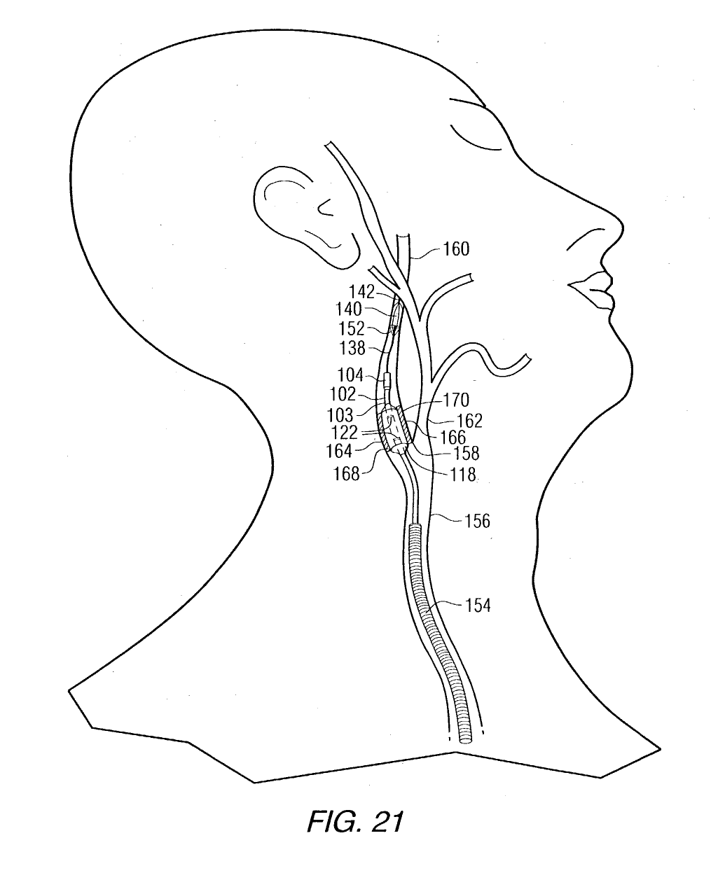

In one embodiment, the catheter 100 may be used to open an occluded blood vessel narrowed by stenosis as shown in FIGS. 21-23. As illustrated in FIG. 21, a guiding sheath 154 may be inserted into a blood vessel such as a common carotid artery 156 located proximal to a bifurcation 158 between an internal carotid artery 160 and an external carotid artery 162. A guide wire 138 containing an embolic filter assembly 140 may be advanced through the guiding sheath 154 and past a stenotic section 164 of the internal carotid artery 160 that is affected by stenosis 166, with the embolic filter assembly 140 in a substantially collapsed position.

The embolic filter assembly 140 may then be opened or expanded. A vascular stent 168 may then be deployed via the guide wire 138 to the location of the stenosis 166. The catheter 100 of the present invention may then be advanced over the guide wire 138 via the guiding sheath 154 and into the internal carotid artery 160. The catheter 100 may be positioned along the guide wire 138 so that the inflatable balloon segment 118 is substantially lined up with the stent 168 and the stenotic section 164 of the internal carotid artery 160. Radiopaque markers 122 located at a portion of the shaft 102 containing the inflatable balloon segment 118 may aid in positioning the inflatable balloon segment 118 relative to the stent 168 and the stenotic section 164 of the internal carotid artery 160. The inflatable balloon segment 118 may then be substantially inflated by supplying any suitable gas or liquid to the inflatable balloon segment 118 via the second port 132, second lumen 128, and shaft apertures 120. As the inflatable balloon segment 118 is substantially inflated, the stenotic section 164 of the internal carotid artery 160 preferably will become dilated and the stent will preferably become effectively embedded into the wall 170 of the internal carotid artery 160. As the stenotic section 164 of the internal carotid artery 160 is dilated with the inflatable balloon segment 118, pieces of stenotic material and other embolic material may become dislodged and may flow through the internal carotid artery 160 and be captured by the expanded embolic filter assembly 140.

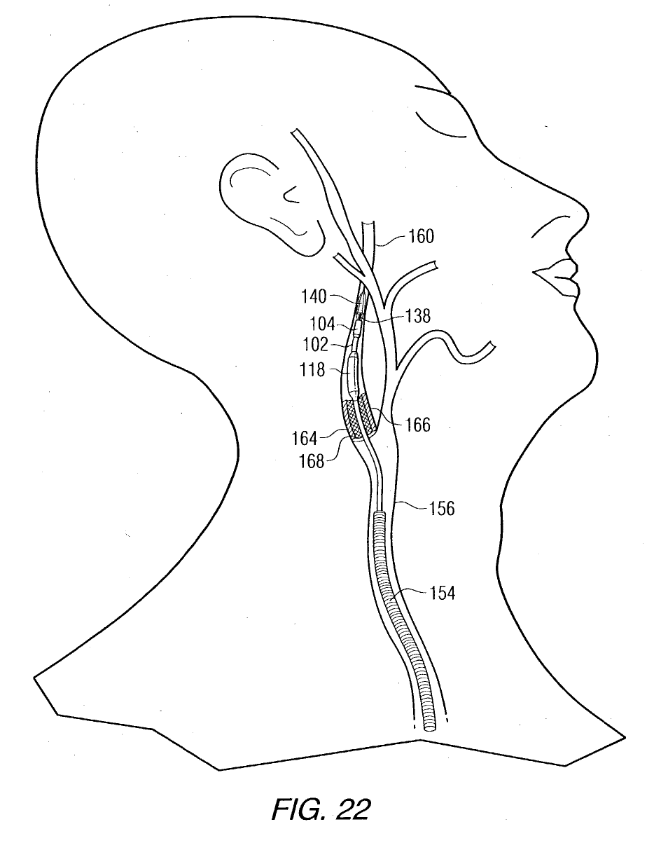

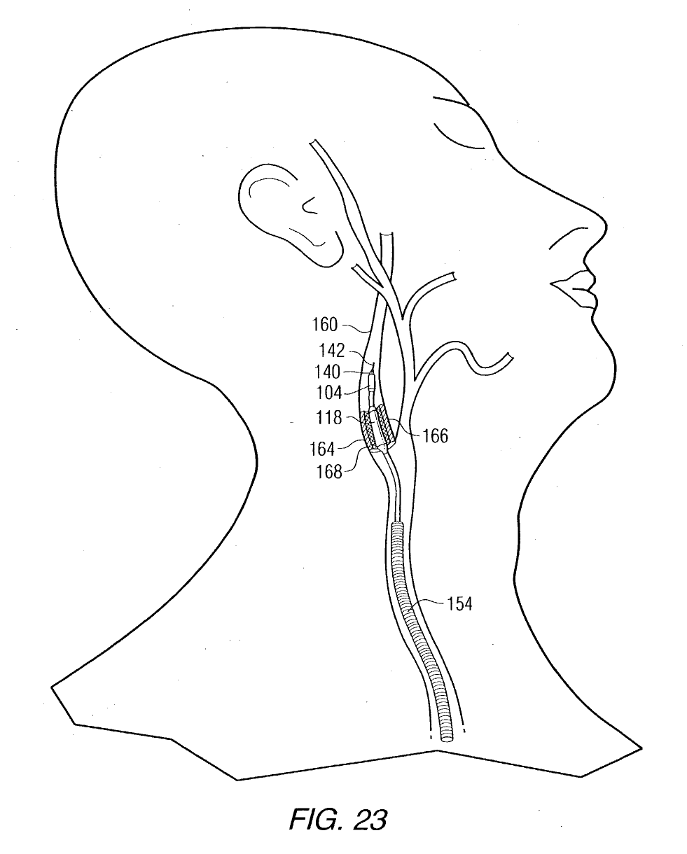

Once the vascular stent 168 is in place, the inflatable balloon segment 118 may be substantially deflated via the second port 132, second lumen 128, and shaft apertures 120, and the catheter 100 may be further advanced coaxially along the guide wire 138 towards the distal end 142 of the guide wire 138. Alternatively, the guide wire 138 may be retracted towards the expanded distal tip 104 of the catheter 100. As shown in FIG. 22, as the expanded distal tip 104 of the catheter 100 meets the control strings 152 of the embolic filter assembly 140, the proximal end (not shown) of the guide wire 138 may be pulled or the catheter 100 may be pushed, preferably causing the embolic filter assembly 140 to begin to collapse into the expanded distal tip 104 of the catheter 100. As shown in FIG. 23, once the embolic filter assembly 140 is substantially collapsed and safely stored within the expanded distal tip 104 of the catheter apparatus 100, the catheter 100 may be retracted into the guiding sheath 154, and the guiding sheath 154 containing the catheter 100 and the potentially debris filled collapsed embolic filter assembly 140 may be removed from the patient as a unit.

It will be appreciated that the catheter apparatus 200 shown in FIGS. 10-12, the catheter apparatus 300 shown in FIGS. 13-16, and the catheter apparatus 400 shown in FIGS. 17-20 may all operate in substantially the same manner as described above.

Whereas particular embodiments of this invention have been described above for purposes of illustration, it will be evident to those skilled in the art that numerous variations of the details of the present invention may be made without departing from the invention as defined in the appended claims.

1. A vascular catheter comprising:
a shaft including an expanded distal tip structured and arranged to receive at least a portion of a thromboembolic protection device; and
a radially expandable segment disposed on the shaft.

2. The vascular catheter of claim 1, wherein the expanded distal tip has an inner cross-sectional diameter that is greater than an inner cross-sectional diameter of an intermediate portion of the shaft.

3. The vascular catheter of claim 2, wherein the ratio of the inner cross-sectional diameter of the expanded distal tip to the inner cross-sectional diameter of the intermediate portion of the shaft is from about 1.6:1 to about 3:1.

4. The vascular catheter of claim 2, wherein the ratio of the inner cross-sectional diameter of the expanded distal tip to the inner cross-sectional diameter of the intermediate portion of the shaft is from about 1.8:1 to about 2.5:1.

5. The vascular catheter of claim 2, wherein the inner cross-sectional diameter of the expanded distal tip is from about 0.8 mm to about 1.8 mm.

6. The vascular catheter of claim 2, wherein the inner cross-sectional diameter of the expanded distal tip is from about 0.95 mm to about 1.65 mm.

7. The vascular catheter of claim 2, wherein the inner cross-sectional diameter of the intermediate portion of the shaft is from about 0.4 mm to about 0.6 mm.

8. The vascular catheter of claim 2, wherein the inner cross-sectional diameter of the intermediate portion of the shaft is from about 0.45 mm to about 0.55 mm.

9. The vascular catheter of claim 1, wherein the expanded distal tip has an outer cross-sectional diameter that is greater than an outer cross-sectional diameter of the intermediate portion of the shaft.

10. The vascular catheter of claim 9, wherein the ratio of the outer cross-sectional diameter of the expanded distal tip to the outer cross-sectional diameter of the intermediate portion of the shaft is from about 1.1:1 to about 1.7:1.

11. The vascular catheter of claim 9, wherein the ratio of the outer cross-sectional diameter of the expanded distal tip to the outer cross-sectional diameter of the intermediate portion of the shaft is from about 1.1:1 to about 1.6:1.

12. The vascular catheter of claim 9, wherein the outer cross-sectional diameter of the expanded distal tip is from about 1.3 mm to about 3.8 mm.

13. The vascular catheter of claim 9, wherein the outer cross-sectional diameter of the expanded distal tip is from about 2 mm to about 3.5 mm.

14. The vascular catheter of claim 9, wherein the outer cross-sectional diameter of the intermediate portion of the shaft is from about 0.9 mm to about 3 mm.

15. The vascular catheter of claim 9, wherein the outer cross-sectional diameter of the intermediate portion of the shaft is from about 1.4 mm to about 3 mm.

16. The vascular catheter of claim 1, wherein the expanded distal tip has an outer cross-sectional diameter that is substantially equal to an outer cross-sectional diameter of the intermediate portion of the shaft.

17. The vascular catheter of claim 1, wherein the ratio of the inner cross-sectional diameter of the expanded distal tip to a length of the expanded distal tip is from about 0.38:1 to about 0.12:1.

18. The vascular catheter of claim 1, wherein the ratio of the inner cross-sectional diameter of the expanded distal tip to a length of the expanded distal tip is from about 0.23:1 to about 0.13:1.

19. The vascular catheter of claim 1, wherein the expanded distal tip has a length ranging from about 0.3 cm to about 1 cm.

20. The vascular catheter of claim 1, wherein the expanded distal tip has a length ranging from about 0.5 cm to about 0.7 cm.

21. The vascular catheter of claim 1, wherein the shaft defines a first lumen running inside the catheter extending substantially to the expanded distal tip.

22. The vascular catheter of claim 21, further comprising a first port in flow communication with the first lumen.

23. The vascular catheter of claim 21, further comprising a guide wire in the first lumen.

24. The vascular catheter of claim 23, wherein the thromboembolic protection device is attached to the guide wire.

25. The vascular catheter of claim 21, wherein the shaft defines a second lumen adjacent to the first lumen extending substantially to the radially expandable segment.

26. The vascular catheter of claim 25 further comprising a second port in flow communication with the second lumen.

27. The vascular catheter of claim 1, wherein the radially expandable segment comprises an inflatable balloon.

28. The vascular catheter of claim 1, wherein the expanded distal tip is substantially cylindrical shaped.

29. The vascular catheter of claim 1, wherein the expanded distal tip is substantially conical shaped.

30. The vascular catheter of claim 1, wherein the expanded distal tip is substantially bulb-shaped.

31. The vascular catheter of claim 1, wherein the shaft includes at least one aperture running radially outward from an inner wall to an outer wall of the shaft.

32. The vascular catheter of claim 31, wherein the shaft includes a plurality of apertures running radially outward from the inner wall to the outer wall of the shaft.

33. The vascular catheter of claim 1, wherein at least a portion of the distal tip includes a flexible material.

34. A catheter assembly comprising:
a shaft including an expanded distal tip;
a radially expandable segment disposed on the shaft; and
a thromboembolic protection device at least partially receivable in the expanded distal tip.

35. The catheter assembly of claim 34, wherein the expanded distal tip has an inner cross-sectional diameter that is greater than an inner cross-sectional diameter of an intermediate portion of the shaft

36. The catheter assembly of claim 35, wherein the ratio of the inner cross-sectional diameter of the expanded distal tip to the inner cross-sectional diameter of the intermediate portion of the shaft is from about 1.6:1 to about 3:1.

37. The catheter assembly of claim 35, wherein the inner cross-sectional diameter of the expanded distal tip is from about 0.8 mm to about 1.8 mm.

38. The catheter assembly of claim 35, wherein the inner cross-sectional diameter of the intermediate portion of the shaft is from about 0.4 mm to about 0.6 mm.

39. The catheter assembly of claim 34, wherein the expanded distal tip has an outer cross-sectional diameter that is greater than an outer cross-sectional diameter of the intermediate portion of the shaft.

40. The catheter assembly of claim 39, wherein the ratio of the outer cross-sectional diameter of the expanded distal tip to the outer cross-sectional diameter of the intermediate portion of the shaft is from about 1.1:1 to about 1.7:1.

41. The catheter assembly of claim 39, wherein the outer cross-sectional diameter of the expanded distal tip is from about 1.3 mm to about 3.8 mm.

42. The catheter assembly of claim 39, wherein the outer cross-sectional diameter of the intermediate portion of the shaft is from about 0.9 mm to about 3 mm.

43. The catheter assembly of claim 34, wherein the expanded distal tip has an outer cross-sectional diameter that is substantially equal to an outer cross-sectional diameter of the intermediate portion of the shaft.

44. The catheter assembly of claim 34, wherein the ratio of the inner cross-sectional diameter of the expanded distal tip to a length of the expanded distal tip is from about 0.38:1 to about 0.12:1.

45. The catheter assembly of claim 34, wherein the expanded distal tip has a length ranging from about 0.3 cm to about 1 cm.

46. The catheter assembly of claim 34, wherein the shaft defines a first lumen running inside the catheter extending substantially to the expanded distal tip.

47. The catheter assembly of claim 46, further comprising a first port in flow communication with the first lumen.

48. The catheter assembly of claim 46, further comprising a guide wire in the first lumen.

49. The catheter assembly of claim 48, wherein the thromboembolic protection device is attached to the guide wire.

50. The catheter assembly of claim 46, wherein the shaft defines a second lumen adjacent to the first lumen extending substantially to the radially expandable segment.

51. The catheter assembly of claim 50 further comprising a second port in flow communication with the second lumen.

52. The catheter assembly of claim 34, wherein the radially expandable segment comprises an inflatable balloon.

53. The catheter assembly of claim 34, wherein the expanded distal tip is substantially cylindrical shaped.

54. The catheter assembly of claim 34, wherein the expanded distal tip is substantially conical shaped.

55. The catheter assembly of claim 34, wherein the expanded distal tip is substantially bulb-shaped.

56. The catheter assembly of claim 34, wherein the shaft includes at least one aperture running radially from the inner wall to the outer wall of the shaft.

57. The catheter assembly of claim 56, wherein the shaft includes a plurality of apertures running radially from an inner wall to an outer wall of the shaft.

58. The catheter assembly of claim 34, wherein at least a portion of the distal tip includes a flexible material.

59. A vascular catheter comprising:
a shaft including an expanded distal tip for storing at least a portion of a thromboembolic protection device; and
a radially expandable segment disposed on the shaft.

60. The vascular catheter of claim 59, wherein the expanded distal tip has an inner cross-sectional diameter that is greater than an inner cross-sectional diameter of an intermediate portion of the shaft.

61. The vascular catheter of claim 60, wherein the inner cross-sectional diameter of the expanded distal tip is from about 0.8 mm to about 1.8 mm.

62. The vascular catheter of claim 60, wherein the inner cross-sectional diameter of the intermediate portion of the shaft is from about 0.4 mm to about 0.6 mm.

63. The vascular catheter of claim 59, wherein the expanded distal tip has an outer cross-sectional diameter that is greater than an outer cross-sectional diameter of the intermediate portion of the shaft.

64. The vascular catheter of claim 63, wherein the outer cross-sectional diameter of the expanded distal tip is from about 1.3 mm to about 3.8 mm.

65. The vascular catheter of claim 63, wherein the outer cross-sectional diameter of the intermediate portion of the shaft is from about 0.9 mm to about 3 mm.

66. The vascular catheter of claim 59, wherein the expanded distal tip has an outer cross-sectional diameter that is substantially equal to an outer cross-sectional diameter of the intermediate portion of the shaft.

67. The vascular catheter of claim 59, wherein the shaft defines a first lumen running inside the catheter extending substantially to the expanded distal tip.

68. The vascular catheter of claim 67, further comprising a guide wire in the first lumen.

69. The vascular catheter of claim 68, wherein the thromboembolic protection device is attached to the guide wire.

70. The vascular catheter of claim 67, wherein the shaft defines a second lumen adjacent to the first lumen extending substantially to the radially expandable segment.

71. The vascular catheter of claim 59, wherein the radially expandable segment comprises an inflatable balloon.

72. A method of dilating blood vessels and protecting a patient from embolic material, the method comprising:
inserting a guide wire including a thromboembolic protection device into a blood vessel and guiding the guide wire and the thromboembolic protection device past a stenotic section of the blood vessel;
expanding the thromboembolic protection device;
guiding a catheter into the blood vessel along the guide wire, wherein the catheter includes a shaft having an expanded distal tip and a radially expandable segment;
expanding the segment to dilate the stenotic portion of the blood vessel;
guiding the catheter further along the guide wire to receive at least a portion of the thromboembolic protection device within the expanded distal tip; and
removing the catheter and the thromboembolic protection device from the blood vessel.

73. The method of dilating blood vessels and protecting a patient from embolic material of claim 72, wherein the expanded distal tip has an inner cross-sectional diameter that is greater than an inner cross-sectional diameter of an intermediate portion of the shaft.

74. The method of dilating blood vessels and protecting a patient from embolic material of claim 72, wherein the expanded distal tip has an outer cross-sectional diameter that is greater than an outer cross-sectional diameter of an intermediate portion of the shaft.

75. The method of dilating blood vessels and protecting a patient from embolic material of claim 72, wherein the expanded distal tip has an outer cross-sectional diameter that is substantially equal to an outer cross-sectional diameter of an intermediate portion of the shaft.

76. A method of dilating blood vessels and protecting a patient from embolic material, the method comprising:
inserting a guide wire including a thromboembolic protection device into a blood vessel and guiding the guide wire and the thromboembolic protection device past a stenotic section of the blood vessel;
expanding the thromboembolic protection device;
guiding a catheter into the blood vessel along the guide wire, wherein the catheter includes a shaft having an expanded distal tip and a radially expandable segment;
expanding the segment to dilate the stenotic portion of the blood vessel;
retracting the guide wire until at least a portion of the thromboembolic protection device is received within the expanded distal tip; and
removing the catheter and the thromboembolic protection device from the blood vessel.

77. The method of dilating blood vessels and protecting a patient from embolic material of claim 76, wherein the expanded distal tip has an inner cross-sectional diameter that is greater than an inner cross-sectional diameter of an intermediate portion of the shaft.

78. The method of dilating blood vessels and protecting a patient from embolic material of claim 76, wherein the expanded distal tip has an outer cross-sectional diameter that is greater than an outer cross-sectional diameter of an intermediate portion of the shaft.

79. The method of dilating blood vessels and protecting a patient from embolic material of claim 76, wherein the expanded distal tip has an outer cross-sectional diameter that is substantially equal to an outer cross-sectional diameter of an intermediate portion of the shaft.
